(19) United States
(12) Patent Application Publication
(10) Pub. No.: US 2018/0049972 A1
(43) Pub. Date: Feb. 22, 2018

(54) CLEANSING COMPOSITION AND METHOD OF MAKING A CLEANSING SOAP

(71) Applicant: Marlin Brasher, Ashville, AL (US)

(72) Inventor: Marlin Brasher, Ashville, AL (US)

(21) Appl. No.: 15/239,598

(22) Filed: Aug. 17, 2016

(51) Int. Cl. A61K 8/97; A61K 8/92; A61Q 19/10; C11D 3/382; C11D 3/384

(52) U.S. Cl. CPC A61K 8/97; A61K 8/925; A61K 8/922; A61K 2800/28; C11D 3/382; C11D 3/384; A61K 2800/805; A61Q 19/10

(57) ABSTRACT

A cleansing composition and a method of making a cleansing soap for removing grease and dirt from the skin or any other surfaces is disclosed. The cleansing composition comprises a predetermined quantity of a chicken fat, a coconut oil, a lard, an olive oil, and a palm oil. The cleansing composition further comprise a mild abrasive such as an oatmeal. A method of making a cleansing soap for removing grease and dirt from the skin is also disclosed. The proposed cleansing composition is capable of removing any dirt or grease without drying the skin. The cleansing soap also helps in quickly removing the complex stains and heavy soils in a single wash of the skin or any other surfaces.

### BACKGROUND

The present invention generally relates to a cleansing composition, more specifically relates to a cleansing soap composition for removing grease and dirt without drying the skin or removing grease and dirt from any other surfaces. The invention further relates to a method of making the cleansing soap for removing grease and dirt from the skin or any other surfaces.

Generally, a personal cleansing soap are useful for heavy duty personal cleansing for removing heavy soils such as oil, grease, clay and the like. The soap composition contains hard abrasives to assist mechanically for the removal of such heavy soils. The hard abrasives in the soap causes damage to the sensitive skin, skin irritation, excessive defatting, or over drying the skin after multiple usage. Further, these kind of soaps reduces the natural sensory feel of the skin.

The soap composition further uses a chemical solvent to remove the grease. Most commercial soaps uses chemical solvents, which are not biodegradable and cause all kinds of havoc in the ecology system. These commercially available soaps in the market will take several days to wear off the grease completely from the skin. Moreover, it requires intensive washing or scrubbing action to remove the dirt or grease from the body or any surfaces.

There exists an option of removing grease in a single wash effectively from the skin by making the soap using both chemicals and mild abrasives. However, this option is faced with an issue of inheriting the disadvantages of the chemicals and mild abrasives in the soap. The soaps are also prepared by using natural oil, but they are ineffective for cleansing the grease completely from the skin. The procurement of natural oil for preparing the soap is labor intensive, which incurs lot of time and cost in large-scale manufacturing of the soap.

Therefore, there is a need in the art for a cleansing composition to provide a means to encounter the heavy soils such as greases gently from the skin or any other surfaces. There is also a need in the art for the cleansing composition that remove grease and dirt from the body quickly, without causing any irritation or drying to the skin.

### SUMMARY

The present invention relates to a cleansing composition for removing grease and dirt from the skin. In an embodiment, the cleansing composition comprises a chicken fat ranges from 15% to 30% by weight, a coconut oil ranges from 20% to 40% by weight, a lard from ranges from 15% to 30% by weight, an olive oil ranges from 5% to 20% by weight; and a palm oil ranges from 5% to 20% by weight. In one embodiment, the cleansing composition may further comprise a mild abrasive. In another embodiment, the mild abrasive is an oatmeal.

In an embodiment, the cleansing composition comprises chicken fat of 20% by weight, coconut oil of 30% by weight, lard of 23% by weight, olive oil of 12% by weight, and palm oil of 12% by weight. In one embodiment, the cleansing composition may further comprise a mild abrasive. In another embodiment, the mild abrasive is an oatmeal.

In an embodiment, the cleansing composition comprises chicken fat of 20.5% by weight, coconut oil of 30.77% by weight, lard of 23.08% by weight, olive oil of 12.82% by weight, and palm oil of 12.82% by weight. In one embodiment, the cleansing composition may further comprise a mild abrasive. In another embodiment, the mild abrasive is an oatmeal.

In an embodiment, a method of making a cleansing soap for removing grease and dirt from the skin. In one embodiment, the first step of the method comprises preparing an oil composition in a predetermined temperature. In one embodiment, the oil composition comprises a chicken fat ranges from 15% to 30% by weight, a coconut oil ranges from 20% to 40% by weight, a lard from ranges from 15% to 30% by weight, an olive oil ranges from 5% to 20% by weight; and a palm oil ranges from 5% to 20% by weight. In an embodiment, a lye-water mixture is prepared in a predetermined temperature in the second step of the proposed method. In one embodiment, the oil composition and lye-water mixture are mixed to produce the cleansing soap in the final step of the method.

In an embodiment, the oil composition comprises chicken fat of 20% by weight, coconut oil of 30% by weight, lard of 23% by weight, olive oil of 12% by weight, and palm oil of 12% by weight. In one embodiment, the oil composition comprises chicken fat of 20.5% by weight, coconut oil of 30.77% by weight, lard of 23.08% by weight, olive oil of 12.82% by weight, and palm oil of 12.82% by weight.

In an embodiment, a solid cleansing soap is prepared by using a sodium hydroxide lye. In one embodiment, a liquid cleansing soap is prepared by using a potassium hydroxide lye. In an embodiment, the oil composition and lye-water mixture are maintained at a temperature range from 100° F. to 115° F. In one embodiment, the oil composition and lye-water mixture are maintained at a temperature 110° F.

In one embodiment, the lye-water mixture comprises a superfat ratio ranges from 2% to 10% by weight for cleansing the skin. The superfat ratio obtained from the lye-water mixture provides moisturizing effect on the skin. In another embodiment, the lye-water mixture comprises a superfat ratio is 0% by weight for cleansing the surface other than skin, such as for household use.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

FIG. 1 shows a method of making a cleansing soap for removing grease according to an embodiment.

### DETAILED DESCRIPTION

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The term “superfat ratio” is defined as the amount of oils, which are not converted to a soap during saponification reaction. The superfat ratio in the soap provides a moisturizing effect on the skin.

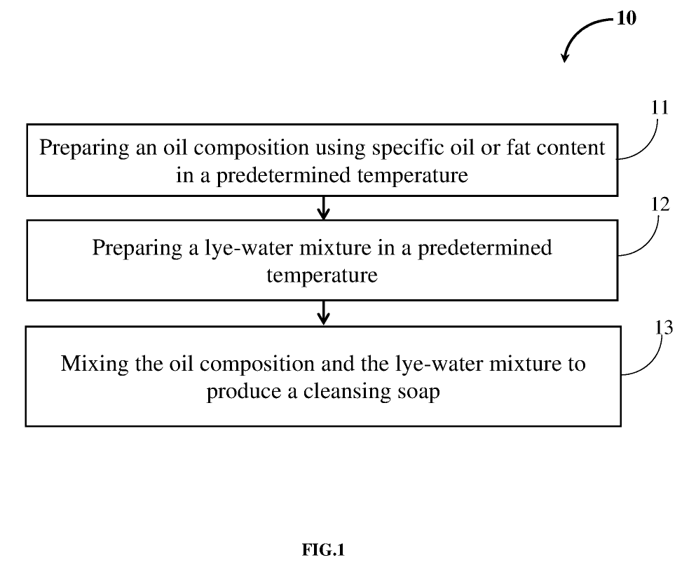

The present invention relates to a cleansing composition and a method of making a cleansing soap for removing grease and dirt from the skin or any other surfaces, such as for any household use. A method 10 of making a cleansing soap for removing grease and dirt from the skin or any other surfaces is shown in FIG. 1. In an embodiment, the method 10 comprises preparing an oil composition using a specific oil or fat content in a predetermined temperature in step 11. In an embodiment, the oil composition comprises a predefined quantity of a chicken fat, a coconut oil, a lard, an olive oil, and a palm oil in step 11. In one embodiment, the oil composition comprises chicken fat ranges from 15% to 30% by weight, coconut oil ranges from 20% to 40% by weight, lard from ranges from 15% to 30% by weight, olive oil ranges from 5% to 20% by weight, and palm oil ranges from 5% to 20% by weight.

In one embodiment, the oil composition comprises chicken fat of 20% by weight, coconut oil of 30% by weight, lard of 23% by weight, olive oil of 12% by weight, and palm oil of 12% by weight. In another embodiment, the oil composition comprises chicken fat of 20.5% by weight, coconut oil of 30.77% by weight, lard of 23.08% by weight, olive oil of 12.82% by weight, and palm oil of 12.82% by weight.

Referring to FIG. 1, the method 10 further comprises an addition of mild abrasive with a minimal quantity in the oil composition of the cleansing soap. In one embodiment, the mild abrasive is an oatmeal. In another embodiment, the mild abrasive is ground oatmeal, which is added to enhance the cleansing performance of the soap. In one embodiment, the quantity of oatmeal is between 0.5 to 2% in the soap.

In an embodiment, the method 10 comprises preparing a lye-water mixture in a predetermined temperature in step 12. In one embodiment, the lye-water mixture comprises a superfat ratio ranges from 2% to 10% by weight for cleansing the skin. The superfat ratio obtained from the lye-water mixture provides moisturizing effect on the skin. In another embodiment, the lye-water mixture comprises a superfat ratio is 0% by weight for cleansing the surface other than skin. In one embodiment, the method 10 for making a solid cleansing soap is prepared by using a sodium hydroxide lye. In another embodiment, the method 10 for making a liquid cleansing soap is prepared by using a potassium hydroxide lye. In one embodiment, the temperature of the oil composition and lye-water mixture are maintained at same or nearly similar parameter such as temperature or pressure.

In an embodiment, the oil composition and lye-water mixture are maintained at a temperature range from 100° F. to 115° F. In one embodiment, the oil composition and lye-water mixture are maintained at a temperature 110° F. In an embodiment, the oil composition and lye-water mixture are mixed to produce the cleansing soap in step 13. In one embodiment, the saponification reaction of the oil or fat composition with the lye forms the cleansing soap. In an embodiment, the temperature of the oil composition and lye-water mixture are maintained at same or nearly similar temperature before mixing in the step 13.

The present invention also relates to a cleansing composition for removing grease and dirt from the skin or any other surfaces, such as for any household use. In an embodiment, the cleansing composition comprises a predetermined quantity of a chicken fat, a coconut oil, a lard, an olive oil, and a palm oil. In one embodiment, the cleansing composition comprises chicken fat ranges from 15% to 30% by weight, coconut oil ranges from 20% to 40% by weight, lard from ranges from 15% to 30% by weight, olive oil ranges from 5% to 20% by weight, and palm oil ranges from 5% to 20% by weight. In one embodiment, the cleansing composition may further comprise a mild abrasive. In another embodiment, the mild abrasive is an oatmeal.

In an embodiment, the cleansing composition comprises chicken fat of 20% by weight, coconut oil of 30% by weight, lard of 23% by weight, olive oil of 12% by weight, and palm oil of 12% by weight. In one embodiment, the cleansing composition may further comprise a mild abrasive. In another embodiment, the mild abrasive is an oatmeal.

In an embodiment, the cleansing composition comprises chicken fat of 20.5% by weight, coconut oil of 30.77% by weight, lard of 23.08% by weight, olive oil of 12.82% by weight, and palm oil of 12.82% by weight. In one embodiment, the cleansing composition may further comprise a mild abrasive. In another embodiment, the mild abrasive is an oatmeal.

In one embodiment, the lye-water mixture comprises a superfat ratio ranges from 2% to 10% by weight for cleansing the skin. The superfat ratio obtained from the lye-water mixture provides moisturizing effect on the skin. In another embodiment, the lye-water mixture comprises a superfat ratio is 0% by weight for cleansing the surface other than skin.

The proposed cleansing composition is capable of removing any dirt or grease without drying the skin. The cleansing soap according to the invention, quickly removes the complex stains and heavy soils such as oil, grease, clay and the like, in a single wash from the skin or any other surfaces. An action of harsh scrubbing, and intensive washing condition could be avoided by using this soap composition. Further, the skin remains soft, gentle, and smooth even after multiple usage of this cleansing soap. The proposed soap will not cause any irritation to any kind of skin, and enhances the sensory feel of the shin. Additionally, the soap is biodegradable, and eco-friendly to the environment.

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A cleansing composition, the composition comprising:
a chicken fat ranges from 15% to 30% by weight;
a coconut oil ranges from 20% to 40% by weight;
a lard from ranges from 15% to 30% by weight;
an olive oil ranges from 5% to 20% by weight; and
a palm oil ranges from 5% to 20% by weight.

2. The cleansing composition of claim 1, comprising chicken fat of 20% by weight, coconut oil of 30% by weight, lard of 23% by weight, olive oil of 12% by weight, and palm oil of 12% by weight.

3. The cleansing composition of claim 1, comprising chicken fat of 20.5% by weight, coconut oil of 30.77% by weight, lard of 23.08% by weight, olive oil of 12.82% by weight, and palm oil of 12.82% by weight.

4. The cleansing composition of claim 1, further comprising a superfat ratio ranges from 2% to 10% by weight.

5. The cleansing composition of claim 1, further comprising a mild abrasive.

6. The cleansing composition of claim 5, wherein the mild abrasive is an oatmeal.

7. A method of making a cleansing soap, the method comprising:
preparing an oil composition in a predetermined temperature, wherein the oil composition comprising:
a chicken fat ranges from 15% to 30% by weight;
a coconut oil ranges from 20% to 40% by weight;
a lard from ranges from 15% to 30% by weight;
an olive oil ranges from 5% to 20% by weight; and
a palm oil ranges from 5% to 20% by weight,
preparing a lye-water mixture in a predetermined temperature; and
mixing the oil composition and lye-water mixture to produce the cleansing soap for removing dirt and grease.

8. The method of claim 7, further comprising an addition of a mild abrasive in the oil composition of the cleansing soap.

9. The method of claim 8, wherein the mild abrasive is an oatmeal.

10. The method of claim 7, wherein the oil composition comprising chicken fat of 20% by weight, coconut oil of 30% by weight, lard of 23% by weight, olive oil of 12% by weight, and palm oil of 12% by weight.

11. The method of claim 7, wherein the oil composition comprising chicken fat of 20.5% by weight, coconut oil of 30.77% by weight, lard of 23.08% by weight, olive oil of 12.82% by weight, and palm oil of 12.82% by weight.

12. The method of claim 7, wherein the lye-water mixture comprises a superfat ratio ranges from 2% to 10% by weight.

13. The method of claim 7, wherein the lye is a sodium hydroxide for preparing a solid cleansing soap.

14. The method of claim 7, wherein the lye is a potassium hydroxide for preparing a liquid cleansing soap.

15. The method of claim 7, wherein the oil composition and lye-water mixture are maintained at a temperature range from 100° F. to 115° F.

16. The method of claim 7, wherein the oil composition and lye-water mixture are maintained at a temperature 110° F.
